(19) United States
(12) Patent Application Publication
(10) Pub. No.: US 2016/0228634 A1
(43) Pub. Date: Aug. 11, 2016

(54) ASEPTIC DRUG DELIVERY SYSTEM AND METHODS

(71) Applicant: Regeneron Pharmaceuticals, Inc., Tarrytown, NY (US)

(72) Inventors: Michael Cupicha, East Schodack, NY (US); Alexei Goraltchouk, Cambridge, MA (US); Mykhaylo Hrytsak, Hastings-on-Hudson, NY (US)

(73) Assignee: Regeneron Pharmaceuticals, Inc., Tarrytown, NY (US)

(21) Appl. No.: 15/017,154

(22) Filed: Feb. 5, 2016

Related U.S. Application Data
(60) Provisional application No. 62/112,716, filed on Feb. 6, 2015.

(51) Int. Cl. A61M 5/14; A61M 5/142

(52) U.S. Cl. CPC A61M 5/1413; A61M 5/14248; A61M 2205/12; A61M 2207/00

(57) ABSTRACT

Aseptic drug delivery systems, drug delivery systems, drug delivery modules, and methods of manufacturing, assembling and using the systems are disclosed. The drug delivery system including a housing, a motion plate coupled to the housing, and a module moveably coupled to the motion plate. The drug delivery module including a plate with a first end and a second end, a cartridge positioned on the plate, and a cover positioned over the cartridge. Methods of manufacturing, assembling and using the aseptic drug delivery system and a method of manufacturing a drug delivery cartridge are also disclosed.

### CROSS REFERENCE TO RELATED APPLICATION

This application claims priority benefit under 35 U.S.C. §119(e) to U.S. provisional application No. 62/112,716 filed Feb. 6, 2015, which is incorporated herein by reference in its entirety.

### FIELD OF THE INVENTION

The present invention relates generally to drug delivery systems for administering medication. More specifically, but not exclusively, the present invention concerns an aseptic drug delivery system.

### BACKGROUND OF THE INVENTION

Currently many auto injectors use glass or rigid plastic vials, syringes, cartridges, or barrels to store the medication for delivery to a patient. Many of these containers include additional parts, such as, flex tubing, transfer IVS, plungers, pistons, stoppers, and the like to push the medication out of the container through a needle for delivery to the patient. These containers are generally coated with chemicals, such as, silicone, to assist the plunger motion during use. The chemicals may be sprayed onto the barrel in liquid form or sprayed as an emulsion and baked onto the glass wall. Although these coatings assist in plunger motion, they cause concerns for chemical compatibility to the medication contained in the containers. In addition, the chemicals and additional components provide additional sources for potential contamination and breakage of the sterile environment. For example, the coatings could leach into the medication, break, or flake off and be injected into the patient.

In addition, many auto injectors use small pieces of tubing to connect the needle to the bottle or container holding the medication. The addition of tubing between the container and needle provides an additional source of potential contamination of the sterile environment. Glass syringes can also introduce contaminates into the drug delivery stream if they chip, crack, or otherwise break during production filling, which may result in glass chards being injected into the patient. Further, anytime a patient or medical professional refills a multiple use auto injector whether by refilling the container or replacing the used cartridge with a pre-filled cartridge, the patient or medical professional must thoroughly clean all components of the system as well as components used to refill the auto injector to maintain a sterile environment. The requirement to sterilize the auto injector system when refilling provides another opportunity for contamination and may also be time consuming and difficult for some patients.

Thus, a drug delivery system that ensures a sterile environment without the risk of contamination is needed.

### SUMMARY OF THE INVENTION

Aspects of the present invention provide an aseptic drug delivery system. The present invention also provides methods for manufacturing, assembling and using the aseptic drug delivery system.

In one aspect provided herein is a drug delivery system that includes a housing, a motion plate coupled to the housing, and a module moveably coupled to the motion plate.

In another aspect, provided herein is a drug delivery module that has a plate with a first end and a second end, a cartridge positioned on the plate, and a cover positioned over the cartridge and coupled to the plate.

In yet another aspect, provided herein is a method of manufacturing a drug delivery cartridge, the method includes obtaining a first cartridge layer and a second cartridge layer. The method also includes forming edge holes in the first cartridge layer and forming a plurality of cavities in the first cartridge layer. The method further includes inserting an injection mechanism into each cavity and forming edge holes in the second cartridge layer. In addition, the method includes aligning the edges holes of the first cartridge layer with the edge holes of the second cartridge layer and sealing the first cartridge layer to the second cartridge layer on at least three edges to form a plurality of cartridges.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a perspective transparent view of an aseptic drug delivery cartridge, in accordance with an aspect of the present invention;

FIG. 2 is a perspective view of the assembled aseptic drug delivery module including the transparent cartridge of FIG. 1, in accordance with an aspect of the present invention;

FIG. 3 is an exploded view of an aseptic drug delivery module including the transparent cartridge of FIG. 1 with a portion of the plate cut away to show the drive plate, in accordance with an aspect of the present invention;

FIG. 4 is an exploded view of an aseptic drug delivery system including the module of FIG. 3, in accordance with an aspect of the present invention;

FIG. 5 is a perspective view of the assembled aseptic drug delivery system of FIG. 4, in accordance with an aspect of the present invention;

FIG. 6 is a top view of a portion of a roll of aseptic drug delivery cartridges, in accordance with an aspect of the present invention; and

FIG. 7 depicts one embodiment of a method of manufacturing an aseptic drug delivery system, in accordance with an aspect of the present invention.

### DETAILED DESCRIPTION FOR CARRYING OUT THE INVENTION

Generally stated, disclosed herein are an aseptic drug delivery cartridge, module, and system. Further, methods of manufacturing, assembling, and using the aseptic drug delivery system are discussed.

In this detailed description and the following claims, the words proximal, distal, anterior, posterior, medial, lateral, superior and inferior are defined by their standard usage for indicating a particular part of a device according to the relative disposition of the device with respect to a body or directional terms of reference. For example, “proximal” means the portion of a device nearest the point of attachment, while “distal” indicates the portion of the device farthest from the point of attachment. As for directional terms, “anterior” is a direction towards the front side of the device, “posterior” means a direction towards the back side of the device, “medial” means towards the midline of the device, “lateral” is a direction towards the sides or away from the midline of the device, “superior” means a direction above and “inferior” means a direction below another object or structure.

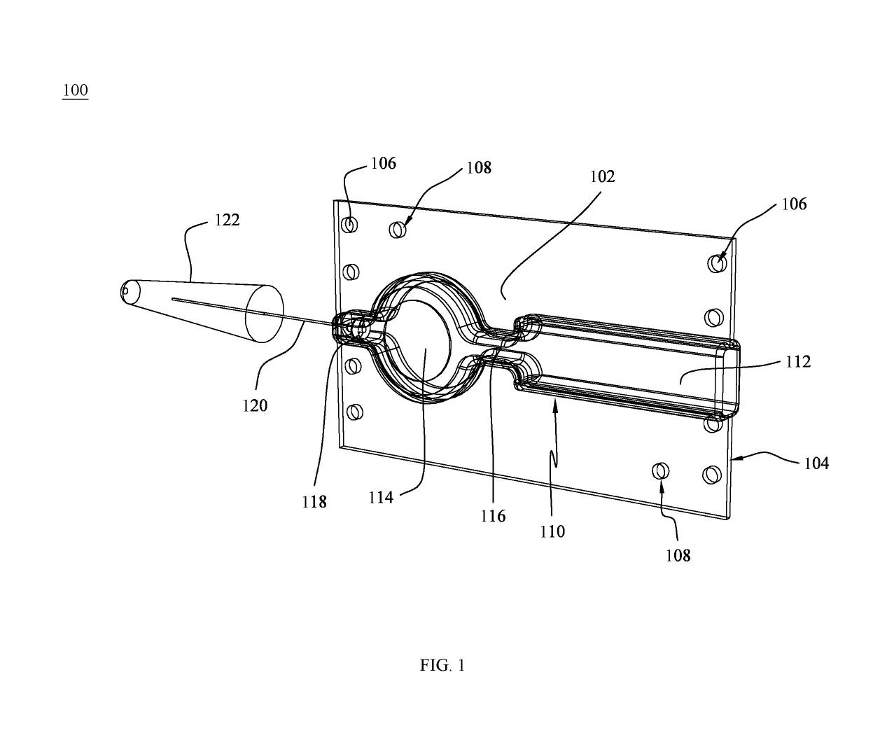

Referring to the drawings, wherein like reference numerals are used to indicate like or analogous components throughout the several views, and with particular reference to FIG. 1, there is illustrated an aseptic drug delivery cartridge 100. The cartridge 100 includes a first layer 102 coupled to a second layer 104. The first and second layers 102, 104 may be, for example, rigid films or flexible films, and the layers 102, 104 may be the same film material or different film materials. The layers 102, 104 may be made of, for example, a number of different polymers based on their chemical compatibility with the medication that will be stored in the cartridges 100 formed of the layers 102, 104. The cartridge 100 may also be, for example, a blow fill seal container, a form fill seal container, or the like. The first layer 102 and second layer 104 may each include a plurality of edge holes 106 extending along the first edges and second edges of the layers 102, 104. The first layer 102 and second layer 104 may each also include fastener holes 108. The fastener holes 108 may include, for example, a first fastener hole 108 positioned near the top edge and a second fastener hole 108 positioned near the bottom edge on each layer 102, 104. The fastener holes 108 may also be positioned, for example, so that the first fastener holes 108 are positioned near the first edge and the second fastener holes 108 are positioned near the second edge. The layers 102, 104 may be attached together by aligning the holes 106, 108 and, for example, heat sealing, gluing, or otherwise bonding the two layers 102, 104 to each other, as described in greater detail below with reference to the method of manufacture. The cartridges 100 may also be formed using, for example, blow fill seal or form fill seal technology.

The cartridge 100 may further include a cavity 110 positioned between the first layer 102 and second layer 104. The cavity 110 may be formed by, for example, the first layer 102 and second layer 104 each being formed to provide half the cavity 110, the first layer 102 being flat and the second layer 104 being formed to create the cavity 110, or the first layer 102 being formed to create the cavity 110 and the second layer 104 being flat. The cavity 110 may be formed by, for example, thermoforming the first layer 102 and/or second layer 104 to create the cavity 110. The cavity 110 may include a first portion 112 and a second portion 114 connected by a pathway 116. The first portion 112 may be, for example, filled with a fluid or medication to be administered to a patient. The second portion 114 may have, for example, at least one flexible surface for moving the medication from the first portion 112 out of the cartridge 100.

It is also contemplated that the first portion 112 of the cavity 110 may, for example, be split into at least two sections, one section may contain a liquid and the other section may contain a second liquid or a powder. The at least two sections of the first portion 112 would have a divider mechanism that could be mechanically removed by, for example, a pull tab or other externally activated mechanism to remove the divider mechanism for mixing of the liquid and powder prior to or during injection into the patient. Alternatively, an external force could be applied to the first portion 112 to break the divider mechanism and allow the at least two sections of the first portion 112 to mix prior to or during injection. The at least two sections could be positioned, for example, parallel to each other and in line with the pathway 116 or in series with each other so one section is at a first end of the first portion 112 and the second section is at a second end of the first portion 112. It is also contemplated that there may be at least two first portions 112 that may either share a pathway 116, second portion 114 and injection mechanism 120 or have separate pathways 116, second portions 114 and injection mechanisms 120 that are adjacent to each other on a single cartridge 100. The first portion 112 of the cavity 110 having at least two sections allows for onsite drug mixing to occur just prior to being administered to the patient. A divided first portion 112 may include, for example, a freeze-dried or lyophilized drug product in the first section, which is reconstituted when mixed with a liquid in a second section of the first portion 112.

The cavity 110 may also include a tip 118 extending out from the second portion 114. The tip 118 may receive an injection mechanism 120, for example, a needle, micro-needle, cannula, or the like. The injection mechanism 120 may be coupled in a fluidic manner to the cavity 110 by, for example, a cannula (not shown). The injection mechanism 120 may be, for example, a needle that may extend out of the cartridge 100 at a first end and the second end of the needle may be fluidically coupled to the cavity 110 by, for example, a cannula. The cartridge 100 may further include a cover or cap 122 for insertion over the injection mechanism 120.

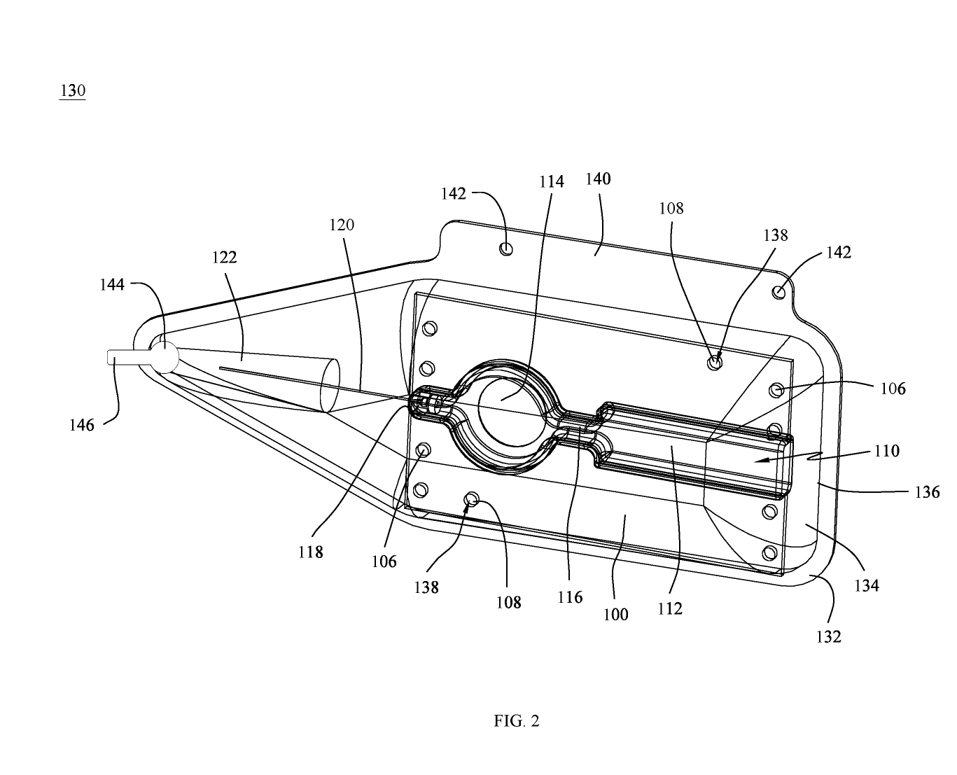

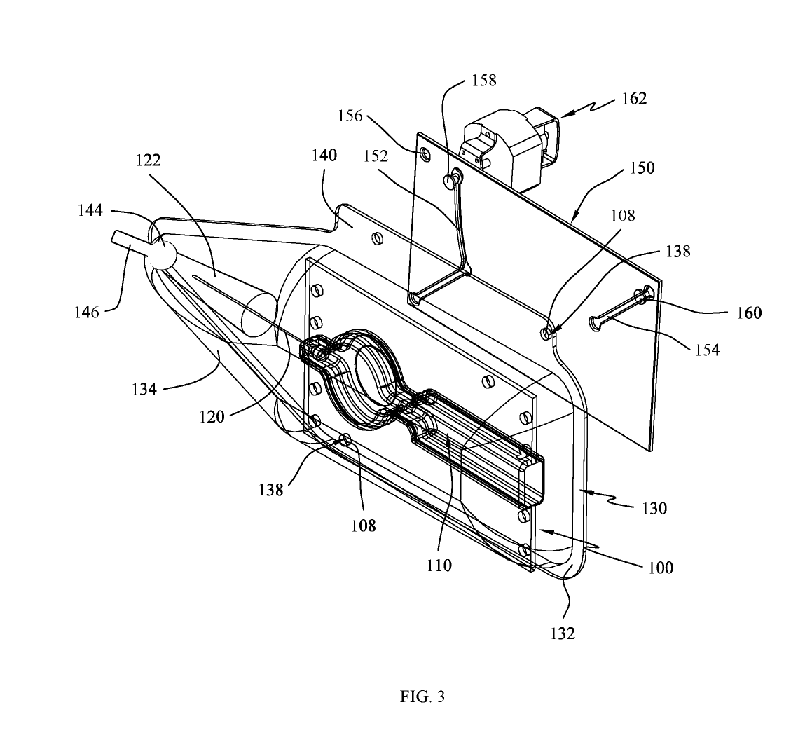

Referring now to FIGS. 2 and 3, with continued reference to FIG. 1, an aseptic drug delivery module 130 including the cartridge 100 is shown. The module 130 also includes a plate 132 and a cover 134. The cover 134 may be made of, for example, a flexible material to allow for the cover 134 to be depressed or crushed to allow for the cartridge 100 to be moved forward and the drug contained in the cartridge 100 to be delivered to a patient. The plate 132 and cover 134 may be, for example, shaped to include a rectangular portion with a triangular portion extending out from the first end. The cartridge 100 is attached to the module 130 by, for example, positioning the cartridge 100 on the plate 132. The cover 134 may then be positioned and applied over the cartridge 100. Once the cover 134 is in the desired position, the cover 134 may be attached to the plate 132 forming a seal 136 around the outside edge of the cover 134. The cover 134 may also be secured to the plate 132 through the fastener holes 108 forming a dot seal 138 to hold the cartridge 100 in the desired position. The plate 132 and cover 134 may form, for example, a sterile housing surrounding the cartridge 100. The plate 132 may also include an extension 140 projecting out from a top side of the plate 132. The extension 140 and the plate 132 may be, for example, planar. The extension 140 may include at least one opening 142. The cover 134 may also include a through hole 144 positioned near the tip of the triangular portion of the cover 134. At least a portion of the cap 122 may extend through the hole 144 in the cover 134. The module 130 may also include a tab 146 which may be positioned over and cover the entire hole 144. The tab 146 may optionally be coupled to the cap 122, such that when the tab 146 is removed, the cap 122 will also be removed to expose the injection mechanism 120.

FIG. 3 also shows a motion plate 150 which the module 130 may be coupled to for injecting medication into a patient. It is also contemplated that the cartridge 100 may be coupled directly to the motion plate 150 with the necessary sterilization of the cartridge 100 and injection mechanism 120 to maintain the same sterile environment as provided by the module 130. The motion plate 150 may include a first track 152 positioned near a first end of the motion plate 150 and a second track 154 positioned near a second end of the motion plate 150. The first track 152 may include, for example, a first portion slanted from a top position near the first end of the motion plate 150 toward a center point of the motion plate 150 and a second portion then slanted toward a bottom position near the first end of the motion plate 150. The second track 154 may be, for example, angled from the second end toward the center point of the motion plate 150. The aseptic drug delivery module 130 may be attached to the motion plate 150 using translating members 158, 160. For example, a first translating member 158 may couple the module 130 to the first track 152 and a second translating member 160 may couple the module 130 to the second track 154. The translating members 158, 160 may slide within the first and second tracks 152, 154, respectively, allowing for the module 130 to move with respect to the motion plate 150. The translating members 158, 160 may be, for example, cams, bar linkages, slides and the like. The motion plate 150 may also include a movement mechanism 162, which is moved proximally in FIG. 3 for ease of illustration. The movement mechanism 162 may be coupled to the translating member 158 to move the module 130 for injection. The movement mechanism 162 may be, for example, a motor, drive spring, cylinder, cable, or the like to move the module 130 along the tracks 152, 154.

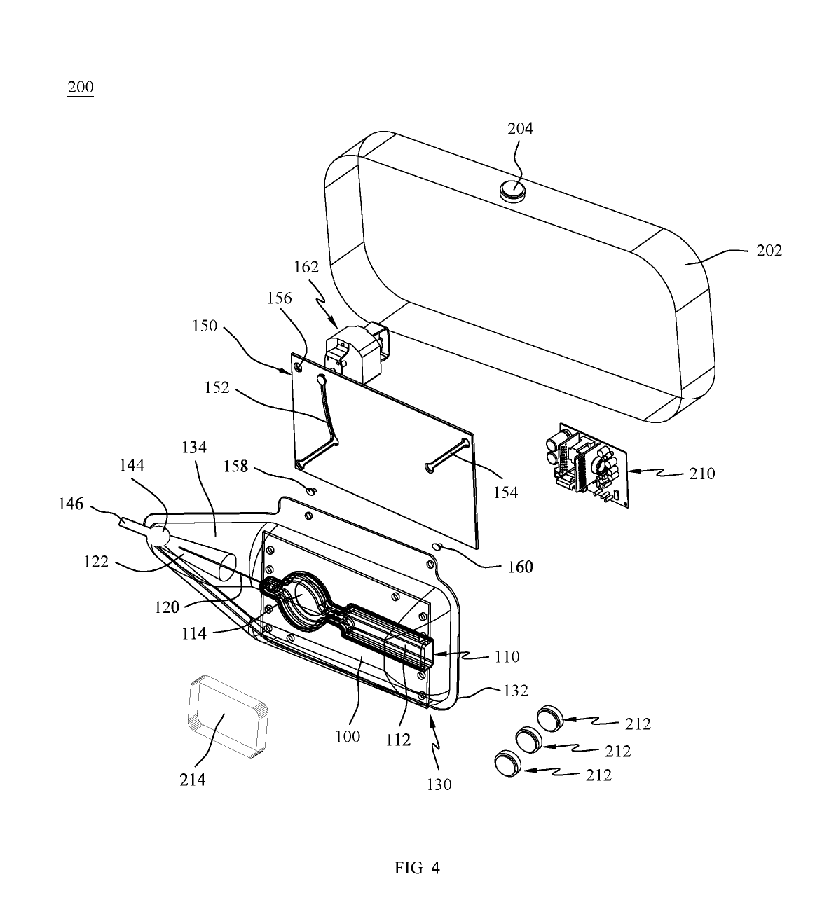

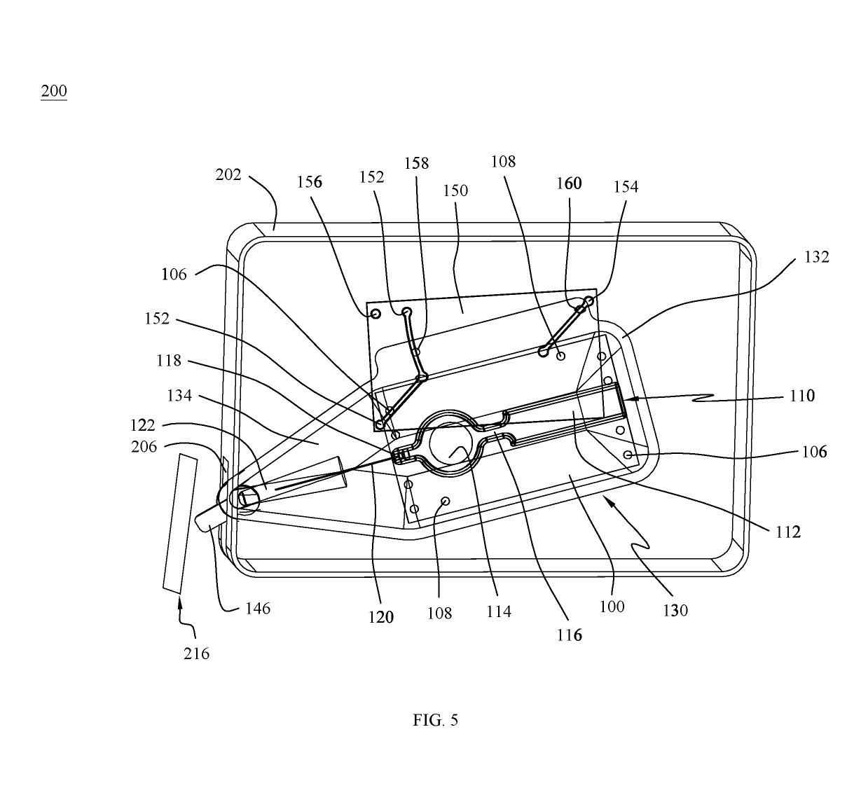

FIGS. 4 and 5 show another embodiment of an aseptic drug delivery system 200. The aseptic drug delivery system 200 may include the motion plate 150 and the aseptic drug delivery module 130 with the aseptic drug delivery cartridge 100. The aseptic drug delivery system 200 may also include a housing with a first portion 202 and a second portion (not shown). The first portion 202 of the housing may also include an activation button 204 to start the medication delivery. The aseptic drug delivery system 200 may further include a controller 210, power source 212, and a delivery mechanism 214. The controller 210 may also be positioned within the housing 102 and may be, for example, a printed circuit board, including a processing circuit, which may also be referred to as a processor and/or a microprocessor. The controller 210 may be coupled to the activation button 204, the movement mechanism 162, and the delivery mechanism 214 for activation and deactivation. The power source 212 may include, for example, at least one battery or other power supply. The delivery mechanism 214 is positioned external to the cavity 110 of the cartridge 100. The delivery mechanism 214 may be, for example, an excitation device, vibrating mechanism, solenoid, magnetic mechanism, plunger, rolling pump, circular pump, stroking pump, peristaltic pump, diaphragm pump, magnetic u-shaped pump, linear pump, constant force springs, motors, rotary actuators, piezo electronics, fluid pressure, and the like.

The first portion 202 and second portion (not shown) may be secured together to form a housing surrounding the cartridge 100, module 130, motion plate 150, movement mechanism 162, controller 210, power source 212, and delivery mechanism 214. The first portion 202 may also include an opening 206 for the injection mechanism 120 to pass through for injection into the patient, as shown in FIG. 5. The aseptic drug delivery system 200 may also optionally include a pull strip 216. The pull strip 216 may be attached over the opening 206 in the first portion 202 of the housing prior to injection. The pull strip 216 may optionally be coupled to the tab 146. The aseptic drug delivery system 200 may be, for example, an aseptic patch pump.

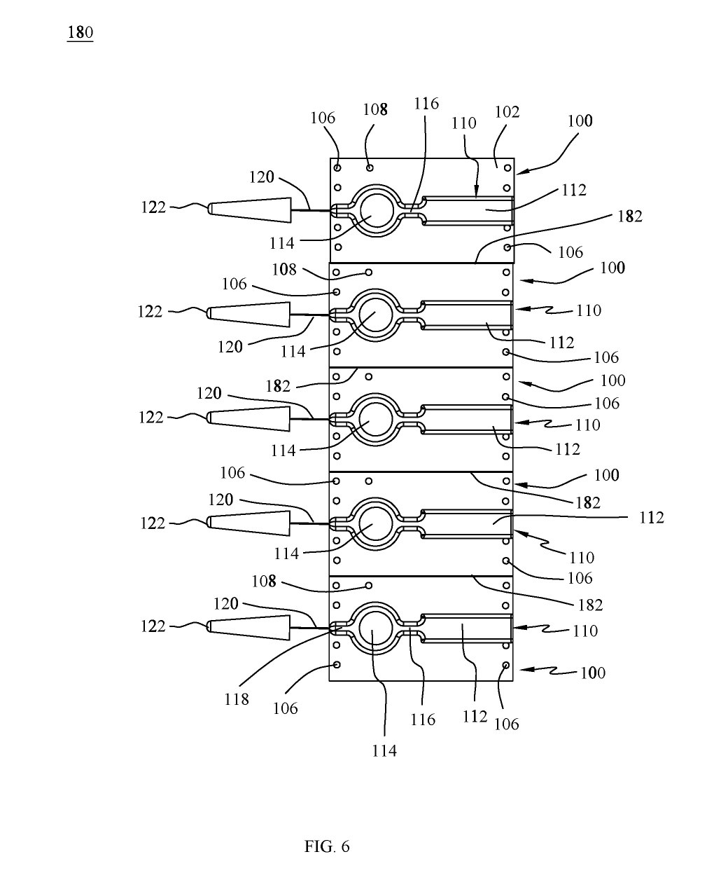

The aseptic drug delivery cartridge 100 may be manufactured as a roll 180 including a plurality of aseptic drug delivery cartridges 100. FIG. 6 shows a portion of the roll 180 of cartridges 100. The cartridges 100 may be, for example, connected at a separation line 182. The separation line 182 may indicate where one cartridge 100 ends and the next cartridge 100 begins. The separation line 182 may also indicate where the cartridges 100 should be separated to form individual cartridges 100. Before separation, the roll 180 may be sterilized, filled with the desired medication, and sealed, as described in greater detail below.

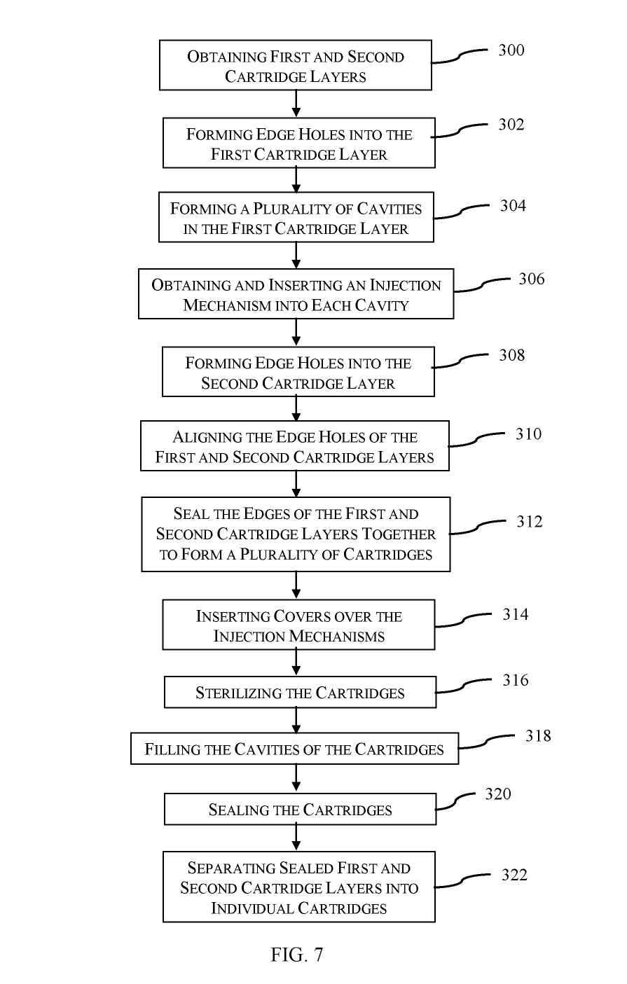

The method of manufacturing the aseptic drug delivery cartridges 100, as shown in FIG. 7, may include obtaining a first cartridge layer and a second cartridge layer 300. The method may also include forming edge holes in the first cartridge layer 302 and forming cavities in the first cartridge layer for receiving medication and the injection mechanisms 304. Injection mechanisms may then be obtained and inserted into each cavity 306. Next, edge holes may be formed in the second cartridge layer 308 and the edge holes of the first and second cartridge layers may be aligned 310. Once the edge holes are aligned the first and second cartridge layers are sealed together to form a plurality of cartridges 312. The first and second layers may be sealed together by, for example, an adhesive or heat. The seal may be formed using, for example, ultrasonic, thermal or other methods to form the seal. Covers may then be placed over the injection mechanisms of each of the plurality of cartridges 314. The plurality of cartridges may be formed into rolls for additional processing. For example, after the cartridges are formed and covers applied, the cartridges may be sterilized 316 and then the cavities of the cartridges filled with the desired fluid or medication 318. After the cartridges are filled, the remaining opening of the cartridge may be sealed to close the cavities 320. Finally, the sealed first and second cartridge layers may be separated into individual cartridges for use 322. If desired or necessary, the filled cartridges may once again be sterilized.

The second cartridge layer may also optionally have cavities formed in the layer prior to aligning the first and second cartridge layers 310 during the method of manufacturing. The individual cartridges 100 each include components that were sterilized prior to being filled with the medication creating an aseptic environment for the medication. As no additional components come into contact with the medication within the cartridges 100 after they are sealed, there is a reduced risk of contamination of the medication while in the cartridges 100 awaiting injection into the patient. Thus, the cartridges 100 maintain an aseptic environment for the medication from the time of filling the cartridges 100 until injection into the patient.

The aseptic drug delivery system 200 may be assembled by obtaining a cartridge 100, a module 130, a motion plate 150, a movement mechanism 162, a housing with a first portion 202 and a second portion, a controller 210, a power source 212, and a delivery mechanism 214. The movement mechanism 162, motion plate 150, controller 210, and power source 212 may all be secured to the interior of the first portion 202 of the housing. The cartridge 100 may be coupled to the module 130 by inserting the translating members 158, 160 through the openings 142 in the plate 132 and the first and second tracks 152, 154, respectively. The translating member 158 may extend through the first track 152 and be moveably secured to a portion of the movement mechanism 162 inside the first housing portion 202. The translating member 160 may extend through the second track 154 and be moveably secured on the back side of the motion plate 150, as shown in FIG. 5. Next, the delivery mechanism 214 may be positioned with respect to the cavity 110 of the cartridge 100 to allow for the delivery mechanism 214 to expel the fluid from the cavity 110. The delivery mechanism 214 may optionally be secured to the second portion (not shown) of the housing. It is also contemplated that the delivery mechanism 214 may be secured to the first housing portion 202 or alternatively, attached directly to the cartridge 100. After all of the internal components of the aseptic drug delivery system 200 are positioned within the first housing portion 202, the second portion (not shown) may be aligned and coupled to the first portion 202 to close the system 200. A pull strip 216, as shown in FIG. 5, may then be applied over the opening 206 in the first housing portion 202. The pull strip 216 may optionally be secured to the tab 146.

Once assembled, the aseptic drug delivery system 200 may be used to deliver a fluid or medication from the cavity 110 to a patient by first positioning the system 200 on the patient's body at the site for administering the medication. The system 200 may be laid or held on the patient's body or alternatively, secured to the patient's body by, for example, an adhesive, bandage wrap, Velcro wrap, or the like. When the patient is ready for administration of the medication, the pull strip 216 may be removed. The pull strip 216 may be attached to the tab 146 which is in turn secured to the cap 122, which allows for the removal of the cap 122 to expose the injection mechanism 120 when the pull strip 216 is removed. Alternatively, the pull strip 216 may not be secured to the tab 146 and after removal of the pull strip 216, the tab 146 would then need to be removed to remove the cap 122 and expose the injection mechanism 120.

Next, the patient, medical professional, or caregiver may push the activation button 204. When the button 204 is pressed it may activate the movement mechanism 162. The movement mechanism 162 in turn moves the module 130 along the tracks 152, 154 to position the injection mechanism 120 for insertion into the patient. For example, the module 130 sliding along tracks 152, 154 may move the module 130 to an insertion position of, for example, approximately 45 degrees, by sliding the first pointed end of the module 130 down the first portion of the track 152 toward the middle of the motion plate 150. Next, the movement mechanism 162 may exert additional force on the module 130 to drive the injection mechanism 120 into the patient at a desired insertion distance. Alternatively, the injection mechanism 120 may be deployed from the system 200 just above the patient's skin for a topical application. To drive the injection mechanism 120 into the patient or position the injection mechanism 120 above the patient's skin, the module 130 is translated down the second portion of the first track 152 at a first end and down the second track 154 at a second end to move the injection mechanism 120 through opening 206 and to or into the patient.

Once the injection mechanism 120 is inserted into the patient or positioned for medication delivery, the delivery mechanism 214 may be activated to move the medication from the first portion 112 of the cavity 110 through the pathway 112 and second portion 114 and out of the tip 118 to the injection mechanism 120 for delivery to the patient. As the medication is delivered to the patient, the cavity 110 may be formed of layers 102, 104 that are elastic to allow the cavity 110 to collapse in upon itself as the pressure changes in the cavity 110. Alternatively, if the cavity 110 is formed of rigid layers 102, 104, the cavity 110 may include, for example, a filter to allow for air to escape from the cavity 110 as the medication is delivered to the patient. The filter, while allowing air to escape, will also prevent fluid from escaping from the cavity 110. In addition, layers 102, 104 of the cavity 110 may include a mechanism to assist with the delivery of the medication, for example, a pumping membrane may be integral to at least one of layers 102, 104 in the second portion 114 of the cavity 110 to allow for the delivery mechanism 214 to pump the medication out of the injection mechanism 120 to the patient.

After the medication delivery is complete, which may be determined based on, for example, sensors which measure time, pressure drop, or the like, the delivery mechanism 214 is turned off. Then, the injection mechanism 120 may be retracted back into the system 200. To retract the injection mechanism 120, the movement mechanism 162 may reverse and slide the module 130 back along the tracks 152, 154 to the starting position where the injection mechanism 120 is positioned entirely within the housing. When retraction of the injection mechanism 120 is complete, the system 200 may optionally send a notification signal to the patient, medical professional, or caregiver, such as a sound indication, light indication, or the like. After the medication delivery is complete, the patient, medical professional or caregiver may remove the system 200 from the patient. If the system 200 is a single use system, the system 200 may then be properly discarded. However, if the system 200 is reusable, the housing may be opened and the empty module 130 may be removed allowing for a new module 130 to be inserted into the system 200. The pull strip 216 may be reinserted over the opening 206 after the new module 130 is inserted into the housing. The pull strip 216 may optionally be attached to the tab 146 of the new module 130. Although the pull strip 216 is preferably reusable, it is also contemplated that the pull strip 216 could be replaceable. The system 200 is then ready for administering the next dosage of medication.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. A drug delivery system, comprising:
a housing;
a motion plate coupled to the housing; and
a module moveably coupled to the motion plate.

2. The drug delivery system of claim 1, further comprising:
a movement mechanism coupled to the housing at a first end and the module at a second end; and
a delivery mechanism positioned adjacent the module.

3. The drug delivery system of claim 1, wherein the motion plate comprises:
a first end and a second end;
a first track positioned near the first end; and
a second track positioned near the second end.

4. The drug delivery system of claim 3, wherein the first track comprises:
a first portion angled from a top position near the first end of the motion plate toward a center point of the motion plate; and
a second portion angled from the center point of the motion plate toward a bottom position near the first end of the motion plate.

5. The drug delivery system of claim 4, wherein the second track is angled from the second end of the motion plate toward the center point of the motion plate.

6. The drug delivery system of claim 5, further comprising:
a first translating member extending through the first track to couple a first end of the module to the movement mechanism; and
a second translating member extending through the second track to moveably coupling a second end of the module to the motion plate.

7. The drug delivery system of claim 1, wherein the module comprises:
a plate with a first end and a second end;
a cartridge positioned on the plate; and
a cover positioned over the cartridge and coupled to the plate.

8. The drug delivery system of claim 1, wherein the housing comprises:
a first portion;
a second portion attached to the first portion;
an activation button extending through the first portion and coupled to the movement mechanism; and
an opening in the first portion of the housing at a first end.

9. The drug delivery system of claim 8, wherein the housing further comprises:
a pull strip attached over the opening in the first portion of the housing.

10. A drug delivery module, comprising:
a plate with a first end and a second end;
a cartridge positioned on the plate; and
a cover positioned over the cartridge.

11. The drug delivery module of claim 7, wherein the cover comprises:
a hole near the first end of the plate.

12. The drug delivery module of claim 8, wherein the cartridge comprises:
an injection mechanism, wherein the injection mechanism extends toward the first end of the plate and through the hole in the cover.

13. The drug delivery module of claim 9, wherein the plate comprises:
an extension projecting out from a top side of the plate; and
at least one opening in the extension.

14. The drug delivery module of claim 10, further comprising:
a tab connected to at least a portion of the cover and at least a portion of the plate, wherein the tab covers the hole in the cover forming a sterile seal.

15. A method of manufacturing a drug delivery cartridge, comprising:
obtaining a first cartridge layer and a second cartridge layer;
forming edge holes in the first cartridge layer;
forming a plurality of cavities in the first cartridge layer;
inserting an injection mechanism into each cavity;
forming edge holes in the second cartridge layer;
aligning the edge holes of the first cartridge layer with the edge holes of the second cartridge layer; and
sealing the first cartridge layer to the second cartridge layer on at least three edges to form a plurality of cartridges.

16. The method of claim 15, further comprising:
inserting a cover over each injection mechanism.

17. The method of claim 16, further comprising:
forming the plurality of cartridges into a roll;
sterilizing the plurality of cartridges;
filling the plurality of cartridges with at least one of a solid and fluid; and
sealing the first cartridge layer to the second cartridge layer on a fourth edge.

18. The method of claim 17, further comprising:
separating the plurality of cartridges into individual cartridges.

19. The method of claim 18, further comprising:
sterilizing the filled cartridges.

20. The method of claim 19, wherein after forming the edge holes in the second cartridge layer, the method further comprises:
forming a plurality of cavities in the second cartridge layer prior to sealing the first cartridge layer to the second cartridge layer.
